(19) United States
(12) Patent Application Publication
(10) Pub. No.: US 2011/0305768 A1
(43) Pub. Date: Dec. 15, 2011

(54) QUICK-DISSOLVING ORAL THIN FILM FOR TARGETED DELIVERY OF THERAPEUTIC AGENTS

(75) Inventors: Hai-Quan Mao, Baltimore, MD (US); Christopher Ku Yu, Shreveport, LA (US); Vu Linh Truong, Campbell, CA (US); Yang Li, San Antonio, TX (US); Dhanya Rangaraj, Foothill Ranch, CA (US); Xuesong Jiang, Baltimore, MD (US); Sagar Ramesh Shah, Clemson, SC (US); Derek Sing, Rockville, MD (US)

(73) Assignee: The Johns Hopkins University, Baltimore, MD (US)

(21) Appl. No.: 13/002,222

(22) PCT Filed: Dec. 24, 2008
(86) PCT No.: PCT/US2008/088300

§ 371 Date: Jun. 14, 2011

Related U.S. Application Data
(60) Provisional application No. 61/133,672, filed on Jul. 1, 2008.

(51) Int. Cl. A61K 9/14; A61K 47/38; A61K 47/32; A61K 47/34; A61K 47/42; A61K 35/76; A61K 35/74; A61K 31/7088; A61K 38/02; A61K 39/395; A61K 38/43; A61K 39/00; A61K 38/21; A61P 37/04; A61P 1/04; A61P 31/00; A61P 1/08; A61P 25/24; A61P 31/10; A61P 29/00; A61P 31/12; A61P 35/00; A61P 37/02; D04H 1/64; B29C 47/14; A61K 47/36; B82Y 5/00

(52) U.S. Cl. 424/499; 424/489; 514/777; 514/781; 514/772.5; 514/772.4; 514/779; 514/772; 514/772.6; 514/778; 514/774; 514/773; 514/775; 424/93.6; 424/93.4; 514/44 R; 514/1.1; 424/130.1; 424/94.1; 424/184.1; 424/85.6; 264/128; 264/460; 977/773; 977/795; 977/915

(57) ABSTRACT

This invention describes a quick-dissolving thin film strips comprising bioactive components encapsulated within pH-sensitive polymeric microparticles. The microparticles are embedded within the thin film and provide protection to components encapsulated within. The invention further describes methods to incorporate bioactive components encapsulated within pH-sensitive polymeric microparticles into a quick-dissolving thin film strip while maintaining the bioactivity of the contained therapeutic agents during thin film formation and microencapsulation.

### CROSS-REFERENCE TO RELATED APPLICATIONS

This application claims priority to U.S. Provisional Application Ser. No. 61/133,672, filed on Jul. 1, 2008, the entire contents of which are incorporated herein by reference.

All references cited herein, whether in print, electronic, computer readable storage media or other form, are expressly incorporated by reference in their entirety and may be employed in the practice of the invention, including but not limited to, abstracts, articles, journals, publications, texts, treatises, technical data sheets, manufacturer's instructions, descriptions, product specifications, product sheets, internet web sites, databases, patents, patent applications, and patent publications.

### FIELD OF THE INVENTION

This invention describes a quick-dissolving thin film strips comprising bioactive components encapsulated within pH-sensitive polymeric microparticles. The microparticles are embedded within the thin film and provide protection to components encapsulated within. The invention further describes methods to incorporate bioactive components encapsulated within pH-sensitive polymeric microparticles into a quick-dissolving thin film strip while maintaining the bioactivity of the contained therapeutic agents during thin film formation and microencapsulation

### BACKGROUND OF THE INVENTION

Oral thin films have been developed for therapeutics destined for delivery in the oral cavity. They are designed to quickly dissolve and release their contents in the oral cavity, initially for breath freshening purposes and dental products. Only recently have oral thin films been identified as potential carriers for more complex components such as typical over-the-counter medications, including dental care and flu medicine. Oral thin films have been identified as a potential alternative to the widely used tablets and liquid drops given orally [3, 5, 8-19]. However, the processes to create these oral thin films are not tailored to package the large variety of therapeutics from bioactive proteins to DNA nanoparticles/gene carriers and live-attenuated viruses. Commercial film manufacturing processes require high temperatures and other extreme conditions that could denature potential biotherapeutic agents and compromise their bioactivity. Furthermore, these oral thin films are primarily designed to deliver therapeutics to the oral cavity, i.e. no further functionality for targeted delivery along the gastrointestinal tract is contained [8-19].

Oral delivery thin-film strips are designed to wet and dissolve quickly upon contact with saliva and buccal tissue, therefore releasing the contained pharmaceutical components. The main component of these thin films is one or more hydrophilic polymers, some of which have good mucoadhesive properties. In such case, the polymeric thin film strongly adheres to buccal tissue until complete dissolution. Quick dissolution and mucoadhesion are key properties important for patient compliance and improved administration of the contained therapeutics [3, 5]. These thin-film strips provide a convenient way to deliver pharmaceutical components (i.e. acetaminophen, dental care products and breath refresher.

For the delivery of drugs where the target tissue is the small intestine, currently available thin-film strips do not provide more functionality than mere convenience. Drugs delivered through the gastrointestinal (GI) tract are subjected to low pH (high acidity) and harsh enzymatic environment in the gastric cavity. Protein drugs, nucleic acids and vaccines are not resistant to these conditions, and are denatured and degraded, leading to significant loss in their bioactivity. Using pH sensitive polymers as a coating to these bioactive components will provide protection in the gastric cavity. The use of pH-sensitive polymers to protect therapeutic agents from gastric acids has been used for many years in oral tablets. The coating of tablet medications with pH-sensitive polymers such as Eudragit® has been shown to be beneficial in providing improved the bioavailability of the swallowed tablets. Furthermore, Eudragit® polymers have also been used to create microcapsules to deliver insulin and other bioactive molecules through the harsh conditions of the gastrointestinal tract [1, 2]. These microcapsules protect its encapsulated compound at the microscale in contrast to the protection of a tablet at the macroscale.

In case of oral delivery of vaccines, targeted delivery to small intestine where the Peyer's patch is located, will not only improve the delivery to antigen presenting cells (M-cells), the efficiency of trans-epithelial transport, but also potentially increase secretory IgA and enhance mucosal immunity, which is most relevant to protection against infections transmitted through mucosal routes [4, 7].

Candidate therapeutics to be packaged in thin films that are preferentially delivered to the small intestines should be “coated” with a protective layer composed of pH-sensitive polymers. Simply embedding such therapeutics into a thin film would only leave them vulnerable to these harsh environments upon ingestion.

Furthermore, there is also a need to maintain product stability through shelf storage after processing into thin films. Maintaining storage stability and simplifying the distribution and administration procedures are critical in order to implement large scale therapeutic and prophylactic treatments. The incorporation of pre-formulated and stabilized drug products, such as in the form of room temperature stable dry powders, into the thin films, could be an added feature of this delivery format resulting in storage stable final dosage presentation

### BRIEF SUMMARY OF THE INVENTION

In one aspect, the invention provides a quick-dissolving thin film composition comprising:
a) one or more water-soluble polymers;
b) one or more mucoadhesive polymers; one or more pH-sensitive microparticles, or mixtures thereof; and
c) one or more bioactive agents
wherein said bioactive agents are indepently encapsulated within said microparticles when said microparticles are present.

In certain aspects, the quick-dissolving thin film composition of the invention comprises one or more mucoadhesive polymers and one or more pH-sensitive microparticles wherein said bioactive agents are indepently encapsulated within said microparticles.

In certain aspects, the quick-dissolving thin film composition of the invention may further comprise one or more pharmaceutically acceptable excipients.

In another aspect, the invention provides a quick-dissolving thin film in which the bioactive agent encapsulated by the pH-sensitive microparticles is a live-attenuated virus, an inactivated virus, a virus like particle, a bacteria, a nucleic acid, a protein, an antibody, an enzyme, an antigen, a growth factor, a cytokine, a small molecular drug or combinations thereof. In other aspects, the bioactive agent encapsulated by the pH-sensitive microparticles is the same in each pH-sensitive microparticle. In other aspects, a quick-dissolving thin film may comprise pH-sensitive microparticle which encapsulate different bioactive agents. In yet other aspects, the invention provides a quick dissolving thin film composition, wherein the bioactive agent is capable of delivering a gene to a subject, including, but not limited to, an adenovirus, an adeno-associate virus, a retrovirus, a paramyxo virus, Salmonella bacteria, Listeria bacteria, Shigella bacteria, E. Coli bacteria, DNA or RNA. In still other aspects, the invention provides a quick-dissolving thin film which further comprises an additional therapeutic agent not encapsulated in the pH-sensitive microparticles.

In another aspect, the pH-sensitive microparticles of the thin film of the invention comprise a copolymer of methacrylic acid or acrylic acid, such as a Eudragit-style copolymer; a pluronic polymer; a chitosan, a chitosan derivative or a combination thereof. In certain aspects, the pH-sensitive microparticles comprise a mixture of Eudragit® L polymer, including, but not limited to Eudragit® L100-55, and Eudragit® S polymer, including, but not limited to, Eudragit® S100. In some aspects, the Eudragit® L polymer and the Eudragit® S polymer is in a weight ratio of about 1:10 to about 10:1, about 1:5 to about 5:1, about 2:3 to about 3:2, or about 3:2. In other aspects, the pH-sensitive microparticles comprise a mixture of Eudragit-style copolymers, Pluronic® F-68 and chitosan or a chitosan derivative. In certain aspects, the weight percentage of Pluronic® F-68 is from about 1% to about 50%, from about 1% to about 25%, or from about 1% to about 20%. In certain aspects, the weight percentage of chitosan or chitosan derivative is from about 1% to about 50%, from about 1% to about 25%, or from about 1% to about 20%.

In another aspect, the pH-sensitive microparticles of the thin film of the invention further comprise a surfactant (including, but not limited to Tween-20 or Tween-80), a sugar (including, but not limited to, manitol or trehalose), a buffering salt (including, but not limited to, potassium phosphate monobasic or potassium phosphate dibasic) or a combination thereof.

In another aspect, the invention provides a quick-dissolving thin film composition comprising:
a) one or more water-soluble polymers;
b) one or more mucoadhesive polymers; one or more pH-sensitive microparticles, or mixtures thereof; and
c) one or more polycation-DNA nanoparticles;
wherein said nanoparticles are indepently encapsulated within said microparticles when said microparticles are present.

In certain aspects, the quick-dissolving thin film composition of the invention comprises one or more mucoadhesive polymers and one or more pH-sensitive microparticles wherein said nanoparticles are indepently encapsulated within said microparticles

In another aspect, the invention provides a method for preparing a quick-dissolving thin film composition comprising one or more pH-sensitive microparticles comprising the steps of:
a. forming an emulsion of one or more bioactive agents, one or more water-soluble polymers and one or more mucoadhesive polymers;
b. dispersing the emulsion into a film forming solution; and
c. forming a film from said dispersion.

In certain aspects, the film of the invention is formed by extrusion or casting onto a flat surface and drying said film under laminar flow, heating or vacuum.

In another aspect, the invention provides a method for preparing a quick-dissolving thin film composition comprising one or more pH-sensitive microparticles comprising the steps of:
a. dispersing pH-sensitive microparticles comprising one or more bioactive agents into a film forming solution; and
b. forming a film from said dispersion.

In certain aspects, the film of the invention is formed by extrusion or casting onto a flat surface and drying said film under laminar flow, heating or vacuum.

In other aspects, the pH-sensitive microparticles comprising one or more bioactive agents are formed by:
a. preparing a suspension or solution of bioactive agents and pH-sensitive polymers;
b. flowing the solution or suspension with a low-pressure gas through a mixing chamber;
c. forming a gaseous suspension of droplets under ultrasonic nozzle conditions; and
d. drying the droplets into powder particles.

In still another aspect, the invention provides a method for preparing a quick-dissolving thin film composition comprising one or more pH-sensitive microparticles comprising the steps of:
a. heating a solution of one or more melt extrudable polymers until melted;
b. mixing the pH-sensitive microparticles with the solution of polymers; and
c. compressing the mixture into a film.
In certain aspects, the solution of polymers is cooled to a temperature that will not melt or otherwise destroy the pH-sensitive microparticles prior to the mixing step.

In still yet another aspect, the invention provides a method for preparing a quick-dissolving thin film composition comprising one or more pH-sensitive microparticles comprising the steps of:
a. electrospinning a first suspension or solution of pH-sensitive polymers optionally comprising one or more bioactive agents to form a mesh; and
b. electrospraying a second suspension or solution of bioactive agents and pH-sensitive polymers to form a film.

In some aspects, the step of electrospinning of the first suspension or solution may be repeated onto the film to produce one or more additional mesh layers over the film.

### BRIEF DESCRIPTION OF THE DRAWINGS

FIG. 1 is an illustration of a representative thin-film strip design.

FIG. 2 is a flow chart representation of the thin-film processing steps and compositions of three phases using Method I as described herein.

FIG. 3 shows two fluorescent microscopic images of microcapsules with encapsulated rhodamine-labeled bovine serum albumin (rh-BSA) prepared by Method I as described herein. Rhodamine-labeled BSA was encapsulated in the microparticles to visualize the particles. Microparticles were retrieved from a thin film strip by dissolving in deionized water (pH5.5).

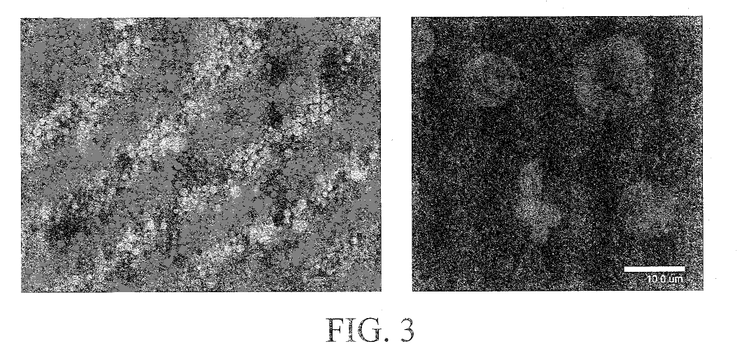

FIG. 4 is a graph of the release of Rhodamine-labeled BSA (rh-BSA) from microparticles retrieved from thin film strips prepared by Method I as described herein in pH 4.0 and pH7.3 buffers, representing the gastric and the small intestinal pH conditions, respectively. Microparticles were suspended in either buffer for various time points. At each time point, the buffer containing released rh-BSA was collected and the fluorescent intensity was measured and correlated to the amount of BSA release.

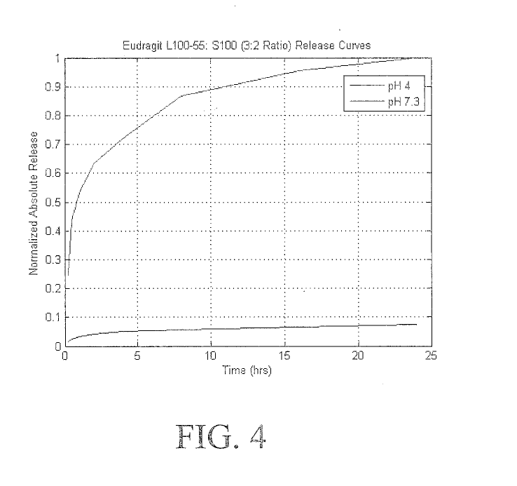

FIG. 5 is an illustrated Schematic diagram showing electrospinning and electrospraying procedure as described in Example VII herein.

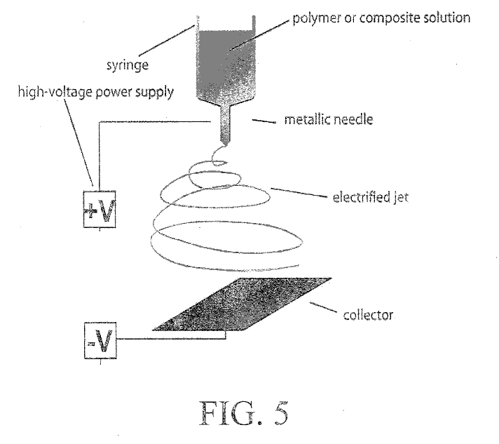

FIG. 6 is a scanning electron micrograph showing the surface of a PVP non-woven mesh film prepared using method described in Example VII herein.

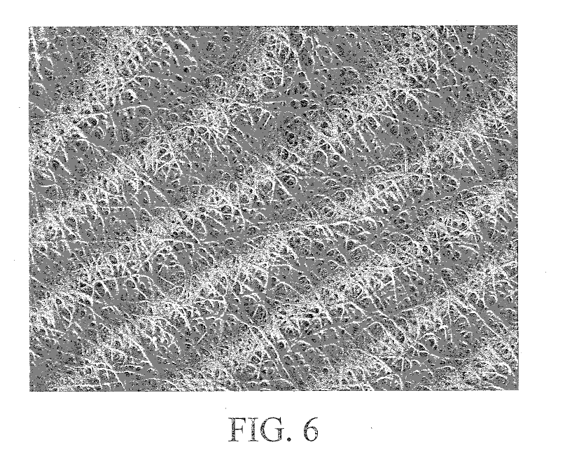

FIG. 7 shows two scanning electron micrographs showing Eudragit microparticle layer encapsulated with Rotavax as described in Example VIII.

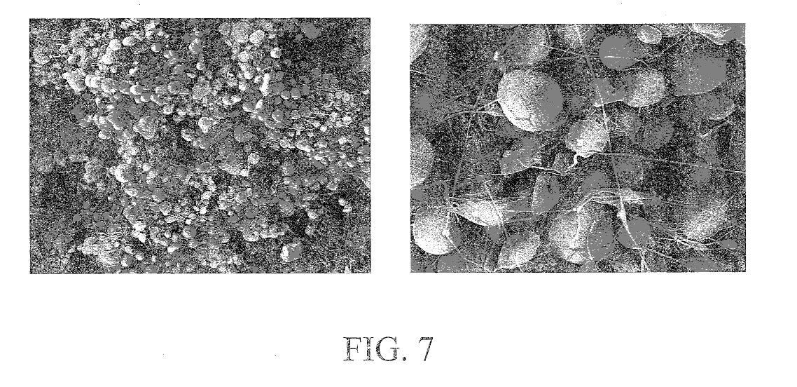

### DETAILED DESCRIPTION OF THE INVENTION

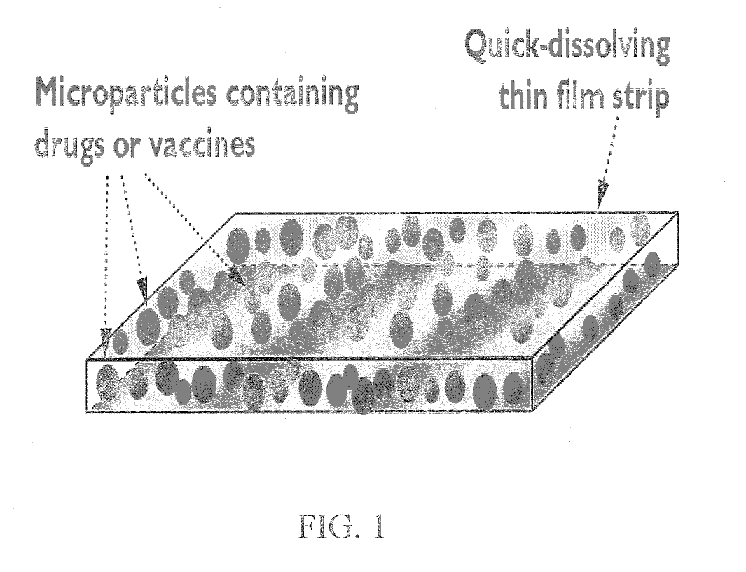

The oral thin film of the invention serves two main functions: quick-dissolving and mucoadhesive properties that enable the film to release the embedded microparticles in oral cavity, pH-sensitive property of the microparticles that enables protection to encapsulated bioactive components in gastric cavity and release them in the small intestine. An additional function is to incorporate preformulated, prestabilized drug products, such as in the form of dry powders, to improve product stability through the film manufacturing process as well as long term storage of the final product. FIG. 1 illustrates the thin film design.

Previously, thin films have not included such functional components that provide protease and pH protection for the bioactive agent to be delivered. The bioactive agent can be coated or encapsulated within nano- and microparticles with the pH-sensitive polymers (polymethacrylates, polyacrylic acids, polyacrylamides, methacrylic acids, cellulose-derivatives and combinations and derivations of these groups) to provide protection. Furthermore, this protection system within our film composition allows for the targeted delivery of the bioactive agent along the gastrointestinal tract upon dissolution in the oral cavity. The use of Eudragit® microparticles for targeted delivery in combination with film-forming polymers is a novel composition for oral thin films.

The oral thin film system has been gaining much attention as an alternative to traditional methods of oral drug delivery such as tablets and liquid droplets. In particular for infants and elderly patients, where swallowing of tablets are difficult and the susceptibility to spitting out the liquid makes traditional methods inconvenient. Oral thin films are designed to be quick-dissolving and mucoadhesive. Mucoadhesion allows the thin film to be retained in the oral cavity until complete dissolution and lowers the chances of spit out, thus potentially improving administration efficiency and patient compliance. While oral thin films are being adopted for use with over-the-counter medications, these thin films remain simple without any higher-order functionality than delivery in the oral cavity. Oral thin films with added functionality, such as that described above, would be more advantageous and preferred for oral delivery of many bioactive agents that are sensitive to acids or enzymes in the gastrointestinal tract.

Thin Film Compositions

In one aspect the invention provides a quick-dissolving thin film composition comprising:
a) one or more water-soluble polymers;
b) one or more mucoadhesive polymers; one or more pH-sensitive microparticles, or mixtures thereof; and
c) one or more bioactive agents
wherein said bioactive agents are independently encapsulated within said microparticles when said microparticles are present.

In certain aspects, the quick-dissolving thin film composition of the invention comprises one or more mucoadhesive polymers and one or more pH-sensitive microparticles wherein said bioactive agents are independently encapsulated within said microparticles.

As used herein, the term “water-soluble polymer” refer to a polymeric composition, soluble in an aqueous solution. Water-soluble polymers useful in the film compositions of the invention may include, but are not limited to pullulan, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, Arabic gum, polyacrylic acid, methylmethacrylate copolymer, carboxyvinyl polymer, amylase, high amylase starch, hydroxypropylated high amylase starch, dextrin, pectin, chitin, chitosan, levan, elsinan, collagen, gelatin, zein, gluten, soy protein isolate, whey protein isolate, and casein.

As used herein, the term “mucoadhesive polymer” refers to a polymer having a good in vivo mucosal absorption rate, safety and degradability. The mucoadhesive polymer used in the present invention may be synthesized or may be naturally-occurring materials. Examples of naturally-occurring mucoadhesive polymers may include, but are not limited to, chitosan, hyaluronate, alginate, gelatin, collagen, and derivatives thereof. Examples of synthetic mucoadhesive polymers may include, but are not limited to, poly(acrylic acid), poly(methacrylic acid), poly(L-lysine), poly(ethyleneimine), poly(ethylene oxide), poly(2-hydroxyethyl methacrylate), and derivatives or copolymers thereof.

As used herein the term “pH-sensitive microparticle” refers to a particle which may encapsulate one or more compounds thereby providing protection to the contents of the microparticle in the gastric cavity. In particular, a pH-sensitive microparticle refers to a particle the solubility of which is dependent on the pH so that it is insoluble in gastric medium but dissolves at some stage after the formulation has emptied from the stomach. Such particles may comprise a copolymer of methacrylic acid or acrylic acid, such as a Eudragit-style copolymer; a pluronic polymer; a chitosan, a chitosan derivative or a combination thereof. The term “Eudragit-sytle copolymer” refers to a polymethacrylate polymer such as, but not limited to Eudragit® L100, Eudragit® S100, Eudragit® RL 100, Eudragit® RS100, Eudragit® E100, Eudragit® L100-55, Eudragit® E PO, Eudragit® RL PO, Eudragit® S PO and the like manufactured by Rohm Co. Ltd. (Germany).

In certain aspects, the pH-sensitive microparticles comprise a mixture of Eudragit® L polymer, including, but not limited to Eudragit® L100-55, and Eudragit® S polymer, including, but not limited to, Eudragit® S100. In some aspects, the Eudragit® L polymer and the Eudragit® S polymer is in a weight ratio of about 1:10 to about 10:1, about 1:5 to about 5:1, about 2:3 to about 3:2, or about 3:2. In other aspects, the pH-sensitive microparticles comprise a mixture of Eudragit-style copolymers, Pluronic® F-68 and chitosan or a chitosan derivative. In certain aspects, the weight percentage of Pluronic® F-68 is from about 1% to about 50%, from about 1% to about 25%, or from about 1% to about 20%. In certain aspects, the weight percentage of chitosan or chitosan derivative is from about 1% to about 50%, from about 1% to about 25%, or from about 1% to about 20%.

In another aspect, the pH-sensitive microparticles of the thin film of the invention further comprise a surfactant (including, but not limited to Tween-20 or Tween-80), a sugar (including, but not limited to, manitol or trehalose), a buffering salt (including, but not limited to, potassium phosphate monobasic or potassium phosphate dibasic) or a combination thereof.

Thin film compositions of the invention can further include solid and edible acids for the maintenance of pH in microparticles. Solid and edible acids include, but are not limited tocitric acid, malic acid, gluconic acid and lactic acid.

In some embodiments of the invention, the buffer for pH control in the microparticles and/or in stability of the active biopharmaceutical ingredient (ABI) can also act as a pH buffer to raise the pH gastric juices when the ABI is administered to an individual. In such a case, it can be preferred that the buffer be at a higher concentration and on the high side of preferred pH values. For example, it can be desirable to have total buffer capacity of the formulation be at least a milliequivalent per liter, (mEq/L), preferably 10 mEq/L or more, 20 mEq/L, 50 mEq/L, 100 mEq/L, 500 mEq/L, 1000 mEq/L, 2000 mEq/L or more. In some embodiments, the buffer capacity can be lower where an antacid is administered separately to the patient in need from administration of the ABI. It is preferred that the buffering capacity of an individual dose to raise a patient's gastric juices range from about 0.5 mEq to 4 mEq, from 0.8 mEq to 2 mEq or about 1 mEq. Preferably, where an individual is to be administered the ABI encased in thin film without a separate buffer composition, the buffer containing thin film provides adequate buffering capacity to raise the individual's gastric cavity to a pH of 4 or higher. The buffer can be e.g., acetate, citrate, succinate, tartarate, maleate, lactate, ammonium bicarbonate, phosphate, magnesium oxide, aluminum oxide, aluminum hydroxide with magnesium hydroxide, aluminum carbonate gel, calcium carbonate, sodium bicarbonate, hydrotalcite, sucralfate, bismuth subsalicylate, and the like.

Thin film compositions of the invention can further include pharmaceutically acceptable excipients and carriers well known in the art, Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or poly(vinylpyrrolidone) (PVP). If desired, disintegrating agents may be added, such as the cross-linked poly(vinyl pyrrolidone), agar, or alginic acid or a salt thereof such as sodium alginate.

Exemplary overall compositions of a typical thin film strip may include:

| Component | Function | Weight % |
|---|---|---|
| Sodium alginate | Film forming, mucoadhesive | 40-50% |
| (low viscosity) | | |
| Polyethyleneoxide | Pliability, mucoadhesive | 10-15% |
| (MW 4000 KDa) | | |
| Polyvinyl alcohol | Pliability, | 20-25% |
| (MW 150 KDa) | adjusting dissolution time, | |
| | Surfactant | |
| Citric acid | Saliva stimulating, maintaining pH | 5-15% |
| Flavor enhancing reagent | Flavor masking | 0.1-2% |
| *Eudragit ® L100-55 | pH-sensitive and targeted delivery | 1-10% |
| *Eudragit ® S100 | Adjust pH-sensitivity | 0.5-5% |
| | and targeted delivery | |
| *Polyols | Stabilizer for bioactive agents | 0.2-2% |
| *Pluronic ® F-68 | Surfactant & adjusting release rate | 0-3% |
| *Tween 20 or Tween 80 | Surfactant | <0.1% |
| *Bioactive components | Therapeutics and vaccines | <0.1% |

*These components are included in the form of microparticles.

The quick-dissolving thin film compositions of the invention may be formed in any shape or size as would be suitable for a particular application. In general, the thin film composition of the present invention is shaped and sized for administration to the oral cavity. In particular, a quick-dissolving thin film composition of the invention may, for example, be in the shape of a rectangle, square, triangle, trapezoid, circle, heart, star, or teardrop shape. Similarly, a quick-dissolving thin film composition of the invention may initially have a thickness of about 500 μm to about 1,500 μm, or about 20 mils to about 60 mils, and when dried have a thickness from about 3 μm to about 250 μm, or about 0.1 mils to about 10 mils. Desirably, the dried firms will have a thickness of about 2 mils to about 8 mils, and more desirably, from about 3 mils to about 6 mils.

Bioactive Materials

The quick-dissolving thin film compositions of the invention comprise pH-sensitive microparticles which encapsulate one or more bioactive materials. Bioactive materials include, but are not limited to live-attenuated viruses, inactivated virus, virus like particles used as vaccines or as delivery vehicles, bacterial vaccines, nucleic acids, proteins, antibodies, enzymes, antigens, growth factors, cytokines, and small molecular drugs or combinations thereof.

The thin film compositions of the invention can comprise a live-attenuated virus, inactivated virus, or a virus like particle used as vaccines or as delivery vehicles. For example, the pH-sensitive microparticle can encapsulate virus vaccines including, but not limited to, Picornaviruses (e.g., polio virus, foot and mouth disease virus), Caliciviruses (e.g., SARS virus, and feline infectious peritonitis virus), Togaviruses (e.g., sindbis virus, the equine encephalitis viruses, chikungunya virus, rubella virus, Ross River virus, bovine diarrhea virus, hog cholera virus), Flaviviruses (e.g., dengue virus, West Nile virus, yellow fever virus, Japanese encephalitis virus, St. Louis encephalitis virus, tick-borne encephalitis virus), Coronaviruses (e.g., human coronaviruses (common cold), swine gastroenteritis virus), Rhabdoviruses (e.g., rabies virus, vesicular stomatitis viruses), Filoviruses (e.g., Marburg virus, Ebola virus), Paramyxoviruses (e.g., measles virus, canine distemper virus, mumps virus, parainfluenza viruses, respiratory syncytial virus, Newcastle disease virus, rinderpest virus), Orthomyxoviruses (e.g., human influenza viruses, avian influenza viruses, equine influenza viruses), Bunyaviruses (e.g., hantavirus, LaCrosse virus, Rift Valley fever virus), Arenaviruses (e.g., Lassa virus, Machupo virus), Reoviruses (e.g., human reoviruses, human rotavirus.), Birnaviruses (e.g., infectious bursal virus, fish pancreatic necrosis virus), Retroviruses (e.g., HIV 1, HIV 2, HTLV-1, HTLV-2, bovine leukemia virus, feline immunodeficiency virus, feline sarcoma virus, mouse mammary tumor virus), Hepadnaviruses (e.g., hepatitis B virus), Parvoviruses (e.g., human parvovirus B, canine parvovirus, feline panleukopenia virus) Papovaviruses (e.g., human papillomaviruses, SV40, bovine papillomaviruses), Adenoviruses (e.g., human adenovirus, canine adenovirus, bovine adenovirus, porcine adenovirus), Herpes viruses (e.g., herpes simplex viruses, varicella-zoster virus, infectious bovine rhinotracheitis virus, human cytomegalovirus, human herpesvirus 6), and Poxviruses (e.g., vaccinia, fowlpoxviruses, raccoon poxvirus, skunkpox virus, monkeypoxvirus, cowpox virus, musculum contagiosum virus).

Those skilled in the art will recognize that compositions or formulas herein relate to viruses that are attenuated by any means, including but not limited to, cell culture passage, reassortment, incorporation of mutations in infectious clones, reverse genetics, other recombinant DNA or RNA manipulation. In addition, those skilled in the art will recognize that other embodiments relate to viruses that are engineered to express any other proteins or RNA including, but not limited to, recombinant flaviviruses, recombinant adenoviruses, recombinant poxviruses, recombinant retroviruses, recombinant adeno-associated viruses and recombinant herpes viruses. Such viruses may be used as vaccines for infectious diseases, vaccines to treat oncological conditions, or viruses to introduce express proteins or RNA (e.g., gene therapy, antisense therapy, ribozyme therapy or small inhibitory RNA therapy) to treat disorders.

In some embodiments, compositions herein can contain one or more viruses with membrane envelopes (e.g., enveloped viruses) of the Togavirus, Flavivirus, Coronavirus, Rhabdovirus, Filovirus, Paramyxovirus, Orthomyxovirus, Bunyavirus, Arenavirus, Retrovirus, Hepadnavirus, Herpesvirus or Poxvirus families. In certain embodiments compositions contain one or more enveloped RNA viruses of the Togavirus, Flavivirus, Coronavirus, Rhabdovirus, Filovirus, Paramyxovirus, Orthomyxovirus, Bunyavirus, Arenavirus, or Retrovirus families. In other embodiments, compositions herein can contain one or more enveloped, positive strand RNA virus of the Togavirus, Flavivirus, Coronavirus, or Retrovirus families. In certain embodiments, compositions can contain one or more live, attenuated Flaviviruses (e.g., dengue virus, West Nile virus, yellow fever virus, or Japanese encephalitis virus).

The thin film compositions of the invention can comprise a live-attenuated or inactivated whole cell bacterial vaccine. For example, the pH-sensitive microparticle can encapsulate bacterial vaccines including, but not limited to, brucella vaccine, pertussis vaccine, plague vaccine, rickettsial vaccines, staphylococcal vaccines, diphtheria-tetanus-pertussis vaccine, haemophilus vaccines, cholera vaccines, anthrax vaccines, lyme disease vaccines, shigella vaccines, escherichia coli vaccines, meningococcal vaccines, diphtheria-tetanus vaccine, streptococcal vaccines, salmonella vaccines, diphtheria-tetanus-acellular pertussis vaccines, tuberculosis vaccines, cholera vaccine, dental caries vaccine, gonorrhea vaccine, haemophilus influenzae vaccine, neisseria meningitidis vaccine, pertussis vaccine, trachoma vaccine and tuberculosis vaccine.

The thin film compositions of the invention can comprise a therapeutic nucleic acid. For example, the pH-sensitive microparticle can encapsulate nucleic acids including, but not limited to, nucleic acids which encode MDA-7, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, ras, MMAC1, FCC, MCC, FUS1, Gene 26 (CACNA2D2), PL6, Beta* (BLU), Luca-1 (HYAL1), Luca-2 (HYAL2), 123F2 (RASSF1), 101F6, Gene 21 (NPRL2), a SEM A3 polypeptide, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Horn/MeI-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-3, MAGE-4, MAGE-5, MAGE-6, p185erbB2, p180erbB-3, c-met, mn-23H1, PSA, TAG-72-4, CA 19-9, CA 72-4, CAM 17.1, NuMa, K-ras, beta-catenin, CDK4, Mum-1, p16, TAGE, PSMA, PSCA, CT7, telomerase, 43-9F, 5T4, 791Tgp72, alpha-fetoprotein, .beta.-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, INGI, mamaglobin, cyclin B1, S100, BRCA1, BRCA2, a tumor immunoglobulin idiotype, a tumor T-cell receptor clonotype, MUC-1, insulin, interferon-alpha, interferon-gamma or epidermal growth factor receptor.

The thin film compositions of the invention can comprise a therapeutic protein. For example, the pH-sensitive microparticle can encapsulate proteins including, but not limited to, human insulin, methionyl-human growth hormone, human insulin analogs, follicle-stimulating hormone, glucagon, human chorionic gonadotropin, human B-type natiuretic peptide, parathyroid hormone, growth hormone analogs, an interferon, EPO, G-CSF, granulocyte/macrophage colony-stimulating factor, an interleukin, consensus interferon, platelet-derived growth factor, an interferon analog, a bone morphogenic protein, human tPA, a modified human tPA, urate oxidase, a blood factor protein, CD3, CD20, a tumor necrosis factor, an HER receptor, CD33, CD52, CD11a, an epidermal growth factor receptor, or a vascular endothelial growth factor.

The thin film compositions of the invention can comprise small interference RNA. For example, the pH-sensitive microparticle can encapsulate an siRNA specific to proteins including, but not limited to, pancreatitis-associated proteins, androgen receptor proteins, VEGF proteins, leukemia fusion proteins, interleukins, or heat shock proteins.

The thin film compositions of the invention can comprise a growth factor. For example, the pH-sensitive microparticle can encapsulate EGF, FGF, GMCSF, HGH, IL-1, PDGF or TGF-8.

The thin film compositions of the invention can comprise a cytokine. Examples of cytokines include, but are not limited to, interleukin-2 (IL-2), interleukin-3 (IL-3), interleukin-4 (IL-4), interleukin-5 (IL-5), interleukin-6 (IL-6), interleukin-7 (IL-7), interleukin-9 (IL-9), interleukin-10 (IL-10), interleukin-12 (IL-12), interleukin 15 (IL-15), interleukin 18 (IL-18), platelet derived growth factor (PDGF), erythropoietin (Epo), epidermal growth factor (EGF), fibroblast growth factor (FGF), granulocyte macrophage stimulating factor (GM-CSF), granulocyte colony stimulating factor (G-CSF), macrophage colony stimulating factor (M-CSF), prolactin, and interferon (IFN), e.g., IFN-alpha, and IFN-gamma).

The thin film compositions of the invention can comprise a small molecular drug. Small molecular drugs include, but are not limited to antibiotics, antiemetic agents, antidepressants, and antifungal agents, anti-inflammatory agents, antiviral agents, anticancer agents, immunomodulatory agents and alkylating agents. Such small molecular drugs are described in further detail below in reference to additional therapeutic agents.

The thin film compositions of the invention can comprise a polycation-DNA nanoparticle. The polycation portion of the nanoparticles may be synthetic or natural. Polycation-DNA nanoparticles include, but are not limited to, chitosan-DNA nanoparticles, PEI-DNA nanoparticles, polyphosphoester-DNA nanoparticles or mixtures thereof.

Additional Therapeutic Agents

Specific compositions of the invention further comprise the an additional therapeutic agent (i.e., a therapeutic agent other than a bioactive agent encapsulated within a pH-sensitive microparticle). Therapeutic agents include, but are not limited to antacids, antibiotics, antiemetic agents, antidepressants, and antifungal agents, anti-inflammatory agents, antiviral agents, anticancer agents, immunomodulatory agents, beta-interferons, hormones or cytokines.

The thin film compositions of the invention can be formulated in combination with antacids. For example, they can be formulated with aluminum carbonate, aluminum hydroxide, bismuth subsalicylate, calcium carbonate, calcium hydroxide, calcium phosphate, dihydroxyaluminum sodium carbonate, magnesium hydroxide, magnesium oxide, magnesium trisilicate, sodium bicarbonate, simethicone, glycine, or combinations thereof.

The thin film compositions of the invention can be formulated in combination with antibiotics. For example, they can be formulated with a macrolide (e.g., tobramycin), a cephalosporin (e.g., cephalexin, cephradine, cefuroxime, cefprozil, cefaclor, cefixime or cefadroxil), a clarithromycin (e.g., clarithromycin), an erythromycin (e.g., erythromycin), a penicillin (e.g., penicillin V) or a quinolone (e.g., ofloxacin, ciprofloxacin or norfloxacin), aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefmetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

The thin film compositions of the invention can be formulated in combination with an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine, tropisetron, and mixtures thereof.

The thin film compositions of the invention can be formulated or formulated in combination with an antidepressant. Suitable antidepressants include, but are not limited to, binedaline, caroxazone, citalopram, dimethazan, fencamine, indalpine, indeloxazine hydrocholoride, nefopam, nomifensine, oxitriptan, oxypertine, paroxetine, sertraline, thiazesim, trazodone, benmoxine, iproclozide, iproniazid, isocarboxazid, nialamide, octamoxin, phenelzine, cotinine, rolicyprine, rolipram, maprotiline, metralindole, mianserin, mirtazepine, adinazolam, amitriptyline, amitriptylinoxide, amoxapine, butriptyline, clomipramine, demexiptiline, desipramine, dibenzepin, dimetacrine, dothiepin, doxepin, fluacizine, imipramine, imipramine N-oxide, iprindole, lofepramine, melitracen, metapramine, nortriptyline, noxiptilin, opipramol, pizotyline, propizepine, protriptyline, quinupramine, tianeptine, trimipramine, adrafinil, benactyzine, bupropion, butacetin, dioxadrol, duloxetine, etoperidone, febarbamate, femoxetine, fenpentadiol, fluoxetine, fluvoxamine, hematoporphyrin, hypericin, levophacetoperane, medifoxamine, milnacipran, minaprine, moclobemide, nefazodone, oxaflozane, piberaline, prolintane, pyrisuccideanol, ritanserin, roxindole, rubidium chloride, sulpiride, tandospirone, thozalinone, tofenacin, toloxatone, tranylcypromine, L-tryptophan, venlafaxine, viloxazine, and zimeldine.

The thin film compositions of the invention can be formulated in combination with an antifungal agent. Suitable antifungal agents include but are not limited to amphotericin B, itraconazole, ketoconazole, fluconazole, intrathecal, flucytosine, miconazole, butoconazole, clotrimazole, nystatin, terconazole, tioconazole, ciclopirox, econazole, haloprogrin, naftifine, terbinafine, undecylenate, and griseofuldin.

The thin film compositions of the invention can be formulated in combination with an anti-inflammatory agent. Useful anti-inflammatory agents include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; steroids including, but not limited to, alclometasone diproprionate, amcinonide, beclomethasone dipropionate, betametasone, betamethasone benzoate, betamethasone diproprionate, betamethasone sodium phosphate, betamethasone valerate, clobetasol proprionate, clocortolone pivalate, hydrocortisone, hydrocortisone derivatives, desonide, desoximatasone, dexamethasone, flunisolide, flucoxinolide, flurandrenolide, halcinocide, medrysone, methylprednisolone, methprednisolone acetate, methylprednisolone sodium succinate, mometasone furoate, paramethasone acetate, prednisolone, prednisolone acetate, prednisolone sodium phosphate, prednisolone tebuatate, prednisone, triamcinolone, triamcinolone acetonide, triamcinolone diacetate, and triamcinolone hexacetonide; and other anti-inflammatory agents including, but not limited to, methotrexate, colchicine, allopurinol, probenecid, sulfinpyrazone and benzbromarone.

The thin film compositions of the invention can be formulated in combination with another antiviral agent. Useful antiviral agents include, but are not limited to, protease inhibitors, nucleoside reverse transcriptase inhibitors, non-nucleoside reverse transcriptase inhibitors and nucleoside analogs. The antiviral agents include but are not limited to zidovudine, acyclovir, gangcyclovir, vidarabine, idoxuridine, trifluridine, and ribavirin, as well as foscarnet, amantadine, rimantadine, saquinavir, indinavir, amprenavir, lopinavir, ritonavir, alpha-interferons; adefovir, clevadine, entecavir, pleconaril.

The thin film compositions of the invention can be formulated in combination with an immunomodulatory agent. Immunomodulatory agents include, but are not limited to, methothrexate, leflunomide, cyclophosphamide, cyclosporine A, mycophenolate mofetil, rapamycin (sirolimus), mizoribine, deoxyspergualin, brequinar, malononitriloamindes (e.g., leflunamide), T cell receptor modulators, and cytokine receptor modulators, peptide mimetics, and antibodies (e.g., human, humanized, chimeric, monoclonal, polyclonal, Fvs, ScFvs, Fab or F(ab)₂ fragments or epitope binding fragments), nucleic acid molecules (e.g., antisense nucleic acid molecules and triple helices), small molecules, organic compounds, and inorganic compounds. Examples of T cell receptor modulators include, but are not limited to, anti-T cell receptor antibodies (e.g., anti-CD4 antibodies (e.g., cM-T412 (Boeringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies, anti-CD11a antibodies (e.g., Xanelim (Genentech)), and anti-B7 antibodies (e.g., IDEC-114 (IDEC)) and CTLA4-immunoglobulin. Examples of cytokine receptor modulators include, but are not limited to, soluble cytokine receptors (e.g., the extracellular domain of a TNF-alpha. receptor or a fragment thereof, the extracellular domain of an IL-1.beta. receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8, IL-9, IL-10, IL-11, IL-12, IL-15, TNF-alpha., interferon (IFN)-alpha., IFN-beta., IFN-gamma, and GM-CSF), anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-alpha. antibodies, anti-IL-1beta antibodies, anti-IL-6 antibodies, anti-IL-8 antibodies (e.g., ABX-IL-8 (Abgenix)), and anti-IL-12 antibodies).

The thin film compositions of the invention can be formulated in combination with cytokines Examples of cytokines include, but are not limited to, interleukin-2 (IL-2), interleukin-3 (IL-3), interleukin-4 (IL-4), interleukin-5 (IL-5), interleukin-6 (IL-6), interleukin-7 (IL-7), interleukin-9 (IL-9), interleukin-10 (IL-10), interleukin-12 (IL-12), interleukin 15 (IL-15), interleukin 18 (IL-18), platelet derived growth factor (PDGF), erythropoietin (Epo), epidermal growth factor (EGF), fibroblast growth factor (FGF), granulocyte macrophage stimulating factor (GM-CSF), granulocyte colony stimulating factor (G-CSF), macrophage colony stimulating factor (M-CSF), prolactin, and interferon (IFN), e.g., IFN-alpha, and IFN-gamma).

The thin film compositions of the invention can be formulated in combination with hormones. Examples of hormones include, but are not limited to, luteinizing hormone releasing hormone (LHRH), growth hormone (GH), growth hormone releasing hormone, ACTH, somatostatin, somatotropin, somatomedin, parathyroid hormone, hypothalamic releasing factors, insulin, glucagon, enkephalins, vasopressin, calcitonin, heparin, low molecular weight heparins, heparinoids, synthetic and natural opioids, insulin thyroid stimulating hormones, and endorphins.

The thin film compositions of the invention can be formulated in combination with beta-interferons which include, but are not limited to, interferon beta-1a and interferon beta-1b.

The thin film compositions of the invention can be formulated in combination with an absorption enhancer, particularly those which target the lymphatic system, including, but not limited to sodium glycocholate; sodium caprate; N-lauryl-D-maltopyranoside; EDTA; mixed micelle; and those reported in Muranishi Crit. Rev. Ther. Drug Carrier Syst., 7-1-33, which is hereby incorporated by reference in its entirety. Other known absorption enhancers can also be used. Thus, the invention also encompasses a pharmaceutical composition comprising one or more sulfated polysaccharides of the invention and one or more absorption enhancers.

The additional therapeutic agent can act additively or, more preferably, synergistically. In a preferred embodiment, a composition comprising a compound of the invention is administered concurrently with the administration of another therapeutic agent, which can be part of the same composition or in a different composition from that comprising the compounds of the invention. In another embodiment, a compound of the invention is administered prior to or subsequent to administration of another therapeutic agent. In a separate embodiment, a compound of the invention is administered to a patient who has not previously undergone or is not currently undergoing treatment with another therapeutic agent, particularly an antiviral agent.

Methods for Processing Thin Film Strips Containing pH-Sensitive Microparticles:

Three processing methods are disclosed to encapsulate bioactive agents either dissolved in aqueous buffer or encapsulated in dry powder and microparticles.

The first is a one-step process to form and embed drug-containing microparticles into a dry oral thin film. This one-step process is an adapted double emulsion solvent evaporation process, in which only one reaction vessel and one drying step are required; and microparticles and the thin film are formed simultaneously. This one-step process is advantageous for industrial considerations as multiple step process to form functional oral thin films would be less attractive than a one step method due to higher costs and complex logistics.

One the other hand, pH-sensitive particles and oral thin films can be formed independently. In case that bioactive agents have been or need to be processed in dry powder form first, the powders or microparticles can be encapsulated in thin film forming solution and processed according to Method II. In dry microparticles, pH-sensitive polymers can be included as a protective and targeted delivery component as discussed above. Other microparticle delivery modifications discussed above can be applied to this process as well.

The third utilizes electrospinning and electrospraying techniques to form thin films and embed the films with bioactive components in pH-sensitive microparticles. This process consists of applying a high voltage to a polymer solution to produce a polymer jet. As the jet travels in air, the jet is elongated under repulsive electrostatic force to produce fibers with diameters in the range of 50 nm to 10 μm, resulting in a random fiber mesh, which then forms a thin film. The properties of the thin film (mechanical properties and dissolution properties) can be controlled by film compositions and film structure (including the layer thickness and number of layers used to form the thin film).

### Method I: Double Emulsion Solvent Evaporation Process to Encapsulate Bioactive Components Dissolved or Suspended in Aqueous Solution and Form a Thin Film.

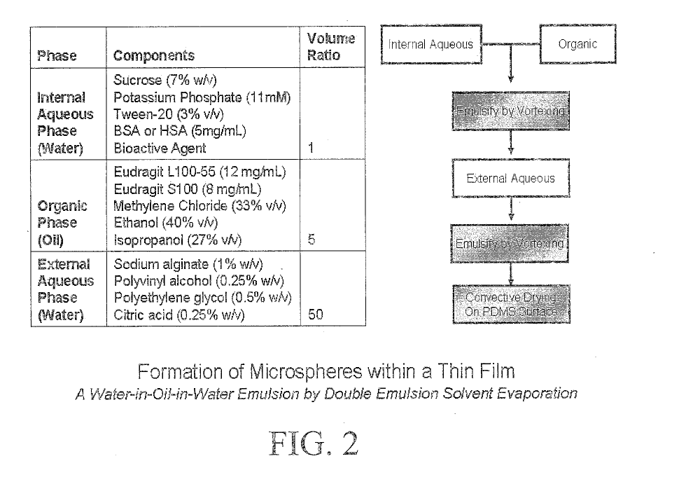

This method combines the microencapsulation and film forming into a one-step process (FIG. 2). It is designed to encapsulate bioactive agents (e.g. proteins, nucleic acids or virus) from their solutions or suspensions.

The process described here is a modified double emulsion solvent evaporation process. Further information on process of this type can be found in Jain D, Majumdar DK and Panda AK., 2006. Three independent phases are used to form the two emulsions: internal aqueous phase, organic phase, external aqueous phase. The compositions of the three phases are:

### Internal Aqueous Phase:

Sucrose: 5-10% w/v

Tween-20: 0.5-5% w/v

Bioactive agents: vaccines, or nucleic acids, protein therapeutics or small molecular drugs

Solvent: potassium phosphate buffer, pH 7.4, 10-25 mM

### Organic Phase:

Eudragit® L100-55: 3-30 mg/mL

Eudragit® S100: 2-20 mg/mL

Pluronic F-68: 0-20 mg/mL

Methylene chloride: 33-70% v/v

Ethanol: 0-50% v/v

Isopropanol: 0-50% v/v

### External Aqueous Phase (Film-Forming Solution):

Sodium alginate (low-viscosity): 1-3% w/v

Polyvinyl alcohol (124,000-186,000 Da, 99% hydrolyzed): 0.25-0.75% w/v

Polyethylene glycol (4,000,000 Da): 0.5-1.5% w/v

Citric acid: 0.25-0.75% w/v

Flavor masking agent: 0.01-0.1% w/v

Solvent: deionized water

One of ordinary skill in the art will recognize that the types and amounts of polymer components described above are exemplary and may be readily modified based on the type and amount of bioactive agent to be formulated or any other factor within in the skill of the ordinary practitioner.

First, the internal aqueous phase, which contains the bioactive agents and the various excipients, is emulsified by vortexing with the organic phase, which contains Eudragit style polymers and excipient polymers to adjust the dissolution time of the microparticles, at a volume ratio of 1:5 to 1:20 (aqueous:organic phase) to form the primary emulsion at 10 to 25° C. The external aqueous phase, containing the film forming polymers and excipients is then emulsified with the primary emulsion at a volume ratio of 50:1 to 5:1 (external aqueous:organic phase) to create the second emulsion by vortexing for 1 to 5 minutes at 10 to 25° C. This double emulsion is then dried on a flat polydimethylsiloxane surface, and dried in a chamber with convective air flow at 10 to 60° C. for 5 to 10 hours. The film is then cut to 2 cm×3 cm or other desired shapes.

### Method II Encapsulation of Dried Microparticles in Thin Film Strips.

Biodegradable or bioabsorbable microparticles containing bioactive components can be directly produced as dried powders using spray drying or lyophilization processes followed by solvent casting or solid dispersion melt/mix method to produce quick dissolving oral thin films. These microparticles can be made to have pH-sensitive properties such that the particles are stable in acidic environment, whereas dissociate, degrade and dissolve in neutral pH. One such example is Eudragit® microparticles (Eudragit L100-55 and Eudragit S100 with a weight ratio of 3:2) that remains solid at pH5.5 or lower and dissolves at pH 7.2 and above. The microparticle production and encapsulation of bioactive components (protein drugs, and DNA complexes) with stabilizing excipients (polyols such as sucrose & trehalose, glycerol, surfactants, small charged amino acids) was prepared using a single process step using ultrasonic spray-drying. The microparticles have the size ranged from 100 nm to 500 μm; preferably in the range of 1 to 10 μm to enhance the uptake in Peyer's patch.

Specifically, the solution that is spray dried is either an aqueous solution or aqueous/organic emulsion.

The aqueous solution composition is as follows:

| Component | Concentration | |
|---|---|---|
| Eudragit ® L100-55 | 0-10 | (w/v) % |
| Eudragit ® S100 | 0-10 | (w/v) % |
| Sucrose | 2-20 | (w/v) % |
| Trehalose | 1-10 | (w/v) % |
| Glycerol | 0.01-0.1 | (v/v) % |
| Pluronic F68 | 0.01-0.1 | (w/v) % |
| Potassium Phosphate | 10-75 | mM |
| Potassium Hydroxide | 0-500 | mM |
| Bioactive Agent | 0-1 | (w/v) % |

| | |
|---|---|
| Solvent | Minimum essential medium |

The aqueous/organic emulsion is as follows:

The aqueous solution:

| Component | Concentration | |
|---|---|---|
| Sucrose | 3-30 | (w/v) % |
| Trehalose | 1.5-15 | (w/v) % |
| Glycerol | 0.01-0.1 | (v/v) % |
| Pluronic F68 | 0.01-0.1 | (w/v) % |
| Potassium Phosphate | 10-100 | mM |
| Bioactive Agent | 0-1 | (w/v) % |

| | |
|---|---|
| Solvent | Minimum essential |
| | medium |

The organic solution:

| Component | Concentration | |
|---|---|---|
| Eudragit ® L100-55 | 0-10 | (w/v) % |
| Eudragit ® S100 | 0-10 | (w/v) % |
| Solvent, Dichloromethane | 33-70 | (v/v) % |
| Solvent, Ethanol | 0-50 | (v/v) % |
| Solvent, Isopropanol | 0-50 | (v/v) % |

One of ordinary skill in the art will recognize that the types and amounts of polymer components described above are exemplary and may be readily modified based on the type and amount of bioactive agent to be formulated or any other factor within in the skill of the ordinary practitioner.

The dry powder microparticles with the loaded bioactive agents (1 to 10 mg) are dispersed in the film-forming solution by vortexing for 1 to 5 minutes at room temperature (solids content 2-20%). The mixture is then cast on a flat polydimethylsiloxane surface and subsequently dried either by air-drying or vacuum-drying at 10 to 60° C. for 5 to 10 hours. The film is then cut to 2 cm×3 cm or other desired shapes.

Method III Electrospin/Electrospray Formation of Thin Film Strips with Embedded Microparticles.

The thin films are formed and embedded with bioactive components in pH-sensitive microparticles through electrospinning and electrospraying techniques by applying a high voltage to a polymer solution to produce a polymer jet. As the jet travels in air, the jet is elongated under repulsive electrostatic force to produce fibers resulting in a random fiber mesh, which then forms a thin film.

The polymer solutions prepared for electrospinning and electrospraying are as follows:

Solution I (for forming quick-dissolving thin film):

| Component | Concentration | |
|---|---|---|
| PVP | 2-40 | (w/v) % |
| Bioactive Agent | 0.5-30 | (w/v) % |
| Solvent, Isopropanol | 30-98 | (v/v) % |

Solution II (for forming pH-sensitive microparticles):

| Component | Concentration | |
|---|---|---|
| Eudragit ® L100-55 | 2-25 | (w/v) % |
| Bioactive Agent | 0.5-30 | (w/v) % |
| Solvent, Isopropanol | 50-98 | (v/v) % |

One of ordinary skill in the art will recognize that the types and amounts of polymer components and the number of polymer layers described herein are exemplary and may be readily modified based on the type and amount of bioactive agent to be formulated or any other factor within in the skill of the ordinary practitioner.

Polymer solution I is loaded into a syringe and placed on a syringe pump. A high-voltage DC power supply is connected to a metal syringe needle and set to between about 5 and about 30 kV. Solution I is then pumped through the needle and polymeric fibers are generated and collected onto a grounded target. The spinning is continued until the fiber mesh reaches about 50 microns to about 1 mm in thickness.

Solution II is then be loaded into a syringe and charged using the same procedure described above. The solution is then electrosprayed to the PVP mesh until the microsphere layer reached 50 to 200 micron thick dependent on the intended loading level of bioactive agent is achieved.

An additional layer of polymer mesh from solution I may be spun onto the first two layers of mesh to increase the thickness of the film if needed.

To aid the spinning process, the collector can be charged with a negative potential of approximately −1 to −10 V. The final film is subsequently vacuum dried or air dried.

### EXAMPLES

One of ordinary skill in the art will recognize that the types and amounts of polymer components described above are exemplary and may be readily modified based on the type and amount of bioactive agent to be formulated or any other factor within in the skill of the ordinary practitioner.

### Example I

### Preparation of a Thin Film Strip (2 Cm×3 Cm×100 μm) Containing Rotavax (Rotaviral Vaccine) Microparticles, Prepared by Double Emulsion Solvent Evaporation Process (Method I)

The following solutions were prepared first:

Phase 1. The internal aqueous phase was created by combining the following:
97 μL Minimal Essential Medium
3 μL Tween-20 (100%)
7 mg sucrose
0.19 mg potassium phosphate (dibasic)
0.5 mg bovine serum albumin
1×10⁷ units of Rotavax (rotavirus)˜1 dosage
Total volume: 100 μL

Phase 2. The organic phase was created by combining the following:
166.5 μL methylene chloride
200.0 μL ethanol
133.5 μL isopropanol
6 mg Eudragit® L100-55
4 mg Eudragit® S100
Total volume: 500 μL

Phase 3. The external aqueous phase was created by combining the following:
50 mg sodium alginate (low-viscosity)
12.5 mg polyvinyl alcohol (124-186 kDa, 99% hydrolyzed)
25 mg polyethylene oxide (4000 kDa)
12.5 mg citric acid
5 mL distilled water
Total volume: 5 mL

The Phase 2 solution was placed in a borosilicate glass vial. The internal aqueous phase (Phase 1 solution) was slowly dripped into the vial while Phase 2 solution was simultaneously vortexed in the vial. This formed the primary emulsion. This primary emulsion was then slowly dripped into Phase 3 solution (the external aqueous phase) while vortexing at 10-25° C. This double emulsion solution was cast onto a flat surface coated with polydimethylsiloxane and constrained to a 2 cm×3 cm surface area. This solution was dried under laminar flow at 20-30° C. for 5-8 hours. The dried solution resulted in a quick-dissolving thin film.

### Example II

### Preparation of a Thin Film Strip (2 Cm×3 Cm×100 μm) Containing Amylase as a Model for Enzyme Therapeutics (Method I)

The following solutions were prepared first:

Phase 1. The internal aqueous phase was created by combining the following:
97 μL Minimal Essential Medium
3 μL Tween-20 (100%)
7 mg sucrose
0.19 mg potassium phosphate (dibasic)
0.5 mg bovine serum albumin
5 mg Amylase
Total volume: 100 μL

Phase 2. The organic phase was created by combining the following:
166.5 μL methylene chloride
200.0 μL ethanol
133.5 μL isopropanol
6 mg Eudragit® L100-55
4 mg Eudragit® S100
Total volume: 500 μL

Phase 3. The external aqueous phase was created by combining the following:
50 mg sodium alginate (low-viscosity)
12.5 mg polyvinyl alcohol (124-186 kDa, 99% hydrolyzed)
25 mg polyethylene oxide (4000 kDa)
12.5 mg citric acid
5 mL distilled water
Total volume: 5 mL

The Phase 2 solution was placed in a borosilicate glass vial. The internal aqueous phase (Phase 1 solution) was slowly dripped into the vial while Phase 2 solution was simultaneously vortexed in the vial. This formed the primary emulsion. This primary emulsion was then slowly dripped into Phase 3 solution (the external aqueous phase) while vortexing at 10-25° C. This double emulsion solution was cast onto a flat surface coated with polydimethylsiloxane and constrained to a 2 cm×3 cm surface area. This solution was dried under laminar flow at 20-30° C. for 5-8 hours. The dried solution resulted in a quick-dissolving thin film.

### Example III

### Preparation of a Thin Film Strip (2 Cm×3 Cm×100 μm) Containing DNA/PEI Nanoparticles (Method I)

The following solutions were prepared first:

Phase 1. The internal aqueous phase was created by combining the following:
97 μL Minimal Essential Medium
3 μL Tween-20 (100%)
7 mg sucrose
0.19 mg potassium phosphate (dibasic)
0.5 mg bovine serum albumin
10 μg DNA/PEI nanoparticles Total volume: 100 μL

Phase 2. The organic phase was created by combining the following:
166.5 μL methylene chloride
200.0 μL ethanol
133.5 μL isopropanol
6 mg Eudragit® L100-55
4 mg Eudragit® S100
Total volume: 500 μL

Phase 3. The external aqueous phase was created by combining the following:
50 mg sodium alginate (low-viscosity)
12.5 mg polyvinyl alcohol (124-186 kDa, 99% hydrolyzed)
25 mg polyethylene oxide (4000 kDa)
12.5 mg citric acid
5 mL distilled water
Total volume: 5 mL

The Phase 2 solution was placed in a borosilicate glass vial. The internal aqueous phase (Phase 1 solution) was slowly dripped into the vial while Phase 2 solution was simultaneously vortexed in the vial. This formed the primary emulsion. This primary emulsion was then slowly dripped into Phase 3 solution (the external aqueous phase) while vortexing at 10-25° C. This double emulsion solution was cast onto a flat surface coated with polydimethylsiloxane and constrained to a 2 cm×3 cm surface area. This solution was dried under laminar flow at 20-30° C. for 5-8 hours. The dried solution resulted in a quick-dissolving thin film

### Example IV

### Preparation of a Thin Film Strip (2 Cm×3 Cm×100 μm) Containing Spray Dried Microparticles with Therapeutic Agents (Method II)

A. Spray Drying Process

A formulation containing a bioactive agent, pH-sensitive polymer and stabilizing excipients was spray dried through an ultrasonic nozzle to create the protective and targeting functions. The formulation to be sprayed was either an aqueous solution or an aqueous/organic emulsion.

The formulation for an aqueous solution is:

| Component | Concentration | |
|---|---|---|
| Eudragit ® L100-55 | 8.33 | (w/v) % |
| Eudragit ® S100 | 8.33 | (w/v) % |
| Sucrose | 17.5 | (w/v) % |
| Trehalose | 7.5 | (w/v) % |
| Glycerol | 0.0625 | (v/v) % |
| Pluronic F68 | 0.05 | (w/v) % |
| Potassium Phosphate | 62.5 | mM |
| Potassium Hydroxide | 416.7 | mM |
| Bioactive Agent (BSA) | 0.5 | (w/v) % |

| | |
|---|---|
| Solvent | Minimum essential medium |

The formulation for an aqueous/organic emulsion is:

The aqueous phase:

| Component | Concentration | |
|---|---|---|
| Sucrose | 26.25 | (w/v) % |
| Trehalose | 11.25 | (w/v) % |
| Glycerol | 0.0938 | (v/v) % |
| Pluronic F68 | 0.075 | (w/v) % |
| Potassium Phosphate | 93.75 | mM |
| Bioactive Agent | 0-1 | (w/v) % |

| | |
|---|---|
| Solvent | Minimum essential medium |

The organic phase:

| Component | Concentration | |
|---|---|---|
| Eudragit ® L100-55 | 10 | (w/v) % |
| Eudragit ® S100 | 10 | (w/v) % |
| Dichloromethane | 40.0 | (v/v) % |
| Ethanol | 33.3 | (v/v) % |
| Isopropanol | 26.7 | (v/v) % |

The formulation was pumped to the ultrasonic nozzle at a flow rate between 0.75-3 mL/min and at a low pressure (5-100 psi), which enabled atomization at low shear stress and spray drying of high solids content and at high viscosity ranges. The formulation was atomized by using a pressurized gas preferably in its supercritical state, while maintaining a small and narrow droplet size distribution. This allowed faster drying of droplets reducing heating stress and minimizing bioactivity loss. A stream of dry, heated gas infused-concurrent to the spray plume dried the formulation forming dried microparticles. The spray drying process is carried out at an ambient temperature of 25° C., humidity of 3%, nozzle temperature of 40-50° C. and a collector temperature of 30-40° C.

B. Formation of the thin film strip.

The spray dried powders were blended with Phase 3 solution as shown in Example I and vortexed briefly at 10-30° C. The suspension was then cast onto a flat surface coated with polydimethylsiloxane and constrained to a 2 cm×3 cm surface area. This solution was dried under laminar flow at 20-30° C. for 5-8 hours to yield a quick-dissolving thin film.

### Example VI

### Release of Microparticles from Film

Each film containing approximately 100-200 mg of polymers and excipients was dissolved in distilled water for 1-2 minutes with vortexing or agitation. Clusters of microparticles were collected by centrifugation at 500-1000 rpm for 1 minute.

Histogram showing the size distribution of Eudragit® particles after complete dissolution of a thin film strip containing these microparticles, confirmed that the majority of the microparticles had diameters ranging from 7 to 10 μm. Size distribution was analyzed using a Z2 Coulter Counter.

### Example VII

### Preparation of a Bi-Layer Film with Rotavax (Rotaviral vaccine) and antacid (MgO)

The following solutions were prepared first:

PVP/Antacid solution was prepared by mixing 100 mg of polyvinyl pyrrolidone (PVP), 100 mg of MgO and 1.5 mL of isopropanol at room temperature.

Rotavax solution was prepared by mixing 1 g of Rotavax (rotavirus) dry powder, 1 g of Eudragit L100-55 and 10.5 mL of isopropanol.

The antacid solution was loaded into a 1-mL syringe and electrospun at 1 mL/hr onto an aluminum foil backing. The film is vacuum-dried for 20 minutes. Subsequently, the Rotavax solution was electrospun at 5 mL/hr over the antacid film until all the solution was consumed. A white film was collected by peeling it from the backing, and cut into appropriate sizes.

### Example VIII

### Preparation of a Bi-Layer Film with Rotavax (Rotaviral Vaccine) Encapsulated in Eudragit Microparticles

The following solutions were prepared first:

PVP solution (10%) was prepared by mixing 1 g of polyvinyl pyrrolidone (PVP) and 10 mL of isopropanol

Eudragit® solution (4%, this can be varied from 2 to 25%, w/v) was prepared by mixing 0.4 g [0.2 g to 2.5 g] of Eudragit® L100-55 and 10 mL of isopropanol

Rotavax solution was prepared by mixing 1 g of Rotavax (rotavirus), 500 μL of isopropanol and 10 mL of Eudragit® solution.

One ml of the 10% (w/v) PVP solution was loaded into a 1-mL syringe and electrospun at 1 mL/hr with +10 kV (5-25 kV) potential charged to the solution and −3 kV applied to the collecting plate (aluminum foil backing). This step can be repeated to achieve the target thickness or loading level. The non-woven mesh film was then vacuum-dried for 20 minutes. The Rotavax solution was loaded into a 1-mL syringe and electrospun at 1 mL/hr and 12 kV over the PVP film. This process can also be repeated to reach the target thickness or loading level for bioactive component. The bi-layered film was then vacuum-dried for 20 minutes, peeled off the aluminum foil, and cut into appropriate sizes.

### EQUIVALENTS

Those skilled in the art will recognize, or be able to ascertain using no more than routine experimentation, many equivalents to the specific embodiments of the invention described herein. Such equivalents are intended to be encompassed by the following claims.

### REFERENCES

1. Jain, D., Majumdar, D. K., Panda, A. K. Insulin Loaded Eudragit L100Microspheres for Oral Delivery: Preliminary in vitro Studies. Journal of Biomaterials Applications. 2006. (2) 21, 195-211.
2. Li, D. Microencapsulation of Protein with Eudragit S100 Polymer. University of Adelaide. Master of Applied Science Engineering Thesis. 2005.
3. Tao, S., Desai, T. A. Gastrointestinal patch systems for oral drug delivery. Drug Discovery Today Reviews. 2005. (10) 13, 909-915.
4. Sanchez, A., Gupta, R., Alonso, M., Siber, G., Langer, R., Pulsed Controlled-Release System for Potential Use in Vaccine Delivery. Pharmaceutical Sciences. 1996. (85) 6, 547-552.
5. Salamat-Miller N., Chittchang, M., Johnston, T. P. The Use of Mucoadhesive Polymers in Buccal Drug Delivery. Advanced F rug Delivery Reviews. 2005. 57, 1666-1691.
6. Weert et. al, Protein Instability in Poly(Lactic-co-Glycolic Acid) Microparticles. Pharmaceutical Research. 2000. 17 (10), 1159-1167.
7. Jones, D. Microencapsulation of Vaccines. Methods in Molecular Medicine Vaccine Protocols. 2003.157-166.
8. U.S. Pat. No. 7,083,781B2: Film Forming Polymers, Methods of Use, and Devices and Applications Thereof
9. U.S. Pat. No. 6,995,137B2: Water-Soluble Natural Film and Its Preparing Method
10. U.S. Pat. No. 7,025,983: Fast dissolving orally consumable films
11. U.S. Pat. No. 6,923,981B2: Fast dissolving orally consumable films
12. U.S. Pat. No. 5,948,430: Water soluble film for oral administration with instant wettability
13. U.S. Pat. No. 6,995,137: Water-soluble natural film and its preparing method
14. U.S. Pat. No. 5,629,003: Rapidly disintegrating sheet-like presentations of multiple dosage units
15. U.S. Pat. No. 6,419,903: Breath freshening film
16. U.S. Pat. No. 6,824,829: Process for manufacturing thin film strips
17. U.S. Pat. No. 7,067,116: Fast dissolving orally consumable solid film containing a taste masking agent and pharmaceutically active agent at weight ratio of 1:3 to 3:1
18. U.S. Pat. No. 7,037,526 B1: Film Preparation for Biphasic Release of Pharmacologically Active or Other Substances
19. USP 2003/0118653: A1 Quick Dissolving Oral Mucosal Drug Delivery Device with Moisture Barrier Coating
20. U.S. Pat. No. 6,552,024: Compositions and methods for mucosal delivery
21. U.S. Pat. No. 6,531,156: Aqueous solvent encapsulation method, apparatus and microcapsules
22. U.S. Pat. No. 7,052,701: Inactivated influenza virus vaccine for nasal or oral application
23. U.S. Pat. No. 5,741,591: Microcapsules, and encapsulation method therefore
24. U.S. Pat. No. 4,710,384: Sustained release tablets made from microcapsules
25. U.S. Pat. No. 5,811,128: Method for oral or rectal delivery of microencapsulated vaccines and compositions therefore
26. U.S. Pat. No. 4,948,586: Microencapsulated insecticidal pathogens
27. U.S. Pat. No. 6,270,800: Aqueous solvent based encapsulation of a bovine herpes virus type-1 subunit vaccine
28. Brayden D. J. and Baird A. W. Microparticle vaccine approaches to stimulate mucosal immunization. Microbes and Infection, 3: 867-876 (2001).

1. A quick-dissolving thin film composition comprising:
a) one or more water-soluble polymers;
b) one or more mucoadhesive polymers; one or more pH-sensitive microparticles, or mixtures thereof; and
c) one or more bioactive agents
wherein said bioactive agents are independently encapsulated within said microparticles when said microparticles are present.

2. The quick-dissolving thin film composition of claim 1 which comprises one or more mucoadhesive polymers and one or more pH-sensitive microparticles wherein said bioactive agents are independently encapsulated within said microparticles.

3. The quick-dissolving thin film composition of claim 1, further comprising one or more pharmaceutically acceptable excipients.

4. The quick dissolving thin film composition of claim 1, wherein the water-soluble polymer is pullulan, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, Arabic gum, polyacrylic acid, methylmethacrylate copolymer, carboxyvinyl polymer, amylase, high amylase starch, hydroxypropylated high amylase starch, dextrin, pectin, chitin, chitosan, levan, elsinan, collagen, gelatin, zein, gluten, soy protein isolate, whey protein isolate, or casein.

5. The quick dissolving thin film composition of claim 4, wherein the water-soluble polymer is polyvinyl pyrrolidone or polyvinyl alcohol.

6. The quick dissolving thin film composition of claim 1, wherein the mucoadhesive polymer is chitosan, hyaluronate, alginate, gelatin, collagen, poly(acrylic acid), poly(methacrylic acid), poly(L-lysine), poly(ethylene imine), poly(ethylene oxide)poly, (2-hydroxyethyl methacrylate) and salts, derivatives or copolymers thereof.

7. The quick dissolving thin film composition of claim 6, wherein the mucoadhesive polymer is sodium alginate or polyethylene oxide or a combination thereof.

8. The quick dissolving thin film composition of claim 1, wherein the bioactive agent is a live-attenuated virus, an inactivated virus, a virus like particle, a bacteria, a nucleic acid, a protein, an antibody, an enzyme, an antigen, a growth factor, a cytokine, a small molecular drug or combinations thereof.

9. The quick dissolving thin film composition of claim 8, wherein the bioactive agent is a live-attenuated virus, an inactivated virus, a virus like particle, or a bacteria.

10. The quick dissolving thin film composition of claim 1, wherein the bioactive agent is capable of delivering a gene to a subject.

11. The quick dissolving thin film composition of claim 10, wherein the bioactive agent capable of delivering a gene is an adenovirus, an adeno-associate virus, a retrovirus, a paramyxo virus, Salmonella bacteria, Listeria bacteria, Shigella bacteria, E. Coli bacteria, DNA or RNA.

12. The quick-dissolving thin film composition of claim 1, wherein all of the pH-sensitive microparticles comprise the same bioactive agent or combination of bioactive agents.

13. The quick-dissolving thin film composition of claim 1, further comprising an additional therapeutic agent not encapsulated in pH-sensitive microparticles.

14. The quick-dissolving thin film composition of claim 13, wherein the additional therapeutic agent is an antacid, an antibiotic, antiemetic agent, antidepressant, antifungal agent, anti-inflammatory agent, antiviral agent, anticancer agent, immunomodulatory agent, beta-interferon, hormone, cytokine or a combination thereof.

15-34. (canceled)

35. A quick-dissolving thin film composition comprising:
a) one or more water-soluble polymers;
b) one or more mucoadhesive polymers; one or more pH-sensitive microparticles, or mixtures thereof; and
c) one or more one or more polycation-DNA nanoparticles;
wherein said nanoparticles are independently encapsulated within said microparticles when said microparticles are present.

36. The quick-dissolving thin film composition of claim 35 which comprises one or more mucoadhesive polymers and one or more pH-sensitive microparticles wherein said nanoparticles are independently encapsulated within said microparticles.

37. A method for preparing a quick-dissolving thin film composition comprising one or more pH-sensitive microparticles comprising the steps of:
a. forming an emulsion of one or more bioactive agents, one or more water-soluble polymers and one or more mucoadhesive polymers;
b. dispersing the emulsion into a film forming solution; and
c. forming a film from said dispersion.

38. (canceled)

39. A method for preparing a quick-dissolving thin film composition comprising one or more pH-sensitive microparticles comprising the steps of:
a. dispersing pH-sensitive microparticles comprising one or more bioactive agents into a film forming solution; and
b. forming a film from said dispersion.

40-41. (canceled)

42. A method for preparing a quick-dissolving thin film composition comprising one or more pH-sensitive microparticles comprising the steps of:
a. heating a solution of one or more melt extrudable polymers until melted;
b. mixing the pH-sensitive microparticles with the solution of polymers; and
c. compressing the mixture into a film.

43. (canceled)

44. A method for preparing a quick-dissolving thin film composition comprising one or more pH-sensitive microparticles comprising the steps of:
a. electrospinning a first suspension or solution of pH-sensitive polymers optionally comprising one or more bioactive agents to form a mesh; and
b. electrospraying a second suspension or solution of bioactive agents and pH-sensitive polymers to form a film.

45. (canceled)
